(19) United States
(12) Patent Application Publication
(10) Pub. No.: US 2025/0381134 A1
(43) Pub. Date: Dec. 18, 2025

(54) AMINO ACID ORAL LIQUID COMPOSITION

(71) Applicant: CENTRE FOR ADDICTION AND MENTAL HEALTH, Toronto (CA)

(72) Inventors: Eduardo FERNANDEZ, Florham Park, NJ (US); Jeffrey MEYER, Toronto (CA)

(21) Appl. No.: 18/876,391

(22) PCT Filed: Jul. 18, 2023
(86) PCT No.: PCT/CA2023/050965

§ 371 Date: Dec. 18, 2024

Related U.S. Application Data
(60) Provisional application No. 63/390,570, filed on Jul. 19, 2022.

(51) Int. Cl. A61K 9/00; A61K 9/08; A61K 31/198; A61K 47/26

(52) U.S. Cl. CPC A61K 9/0095; A61K 9/08; A61K 31/198; A61K 47/26

(57) ABSTRACT

An oral liquid composition comprising an effective amount of an amino acid selected from L-tyrosine and L-tryptophan; a carbohydrate, water, and optionally one or more acceptable excipients, wherein the carbohydrate is at a % by weight of at least 5% w/w with respect to the total weight of composition, and the weight ratio between the amino acid and the carbohydrate is from 1:1 to 1:50. Methods for treatment or prophylaxis of postpartum blues or depression using the oral liquid composition are described.

### CROSS-REFERENCE TO RELATED APPLICATIONS

This application claims the benefit of and claims priority to U.S. Provisional Patent Application No. 63/390,570 entitled AMINO ACID ORAL LIQUID COMPOSITION filed Jul. 19, 2022, the entirety of which is herein incorporated by reference.

### FIELD OF INVENTION

This invention relates to oral liquid compositions comprising an amino acid selected from the group of L-tryptophan and L-tyrosine, the liquid pharmaceutical composition being suitable for oral administration.

### BACKGROUND OF THE INVENTION

Amino acids are essential dietary constituents that are obtained from the proteins contained by food. Among the amino acids, L-tryptophan and L-tyrosine are unique since they are precursors of brain neurotransmitters, the synthesis and release of which is sensitive to relatively small, physiologic changes in precursor concentrations (Fernstrom 1983).

L-Tryptophan is an indispensable amino acid in humans, which in addition to its role in protein synthesis, also participates in complex metabolic pathways where it acts as a precursor to the potent neurotransmitter serotonin, the hormone melatonin, and the vitamin niacin (vitamin B3). L-tryptophan is available from a wide variety of protein-rich foods in the normal diet, including meat, fish, milk and dairy products, egg, beans, lentils and also bread and grains, pasta, rice, fruit and vegetables. L-tyrosine is an essential precursor for the synthesis of the catecholamines neurotransmitters adrenaline (epinephrine), noradrenaline (norepinephrine), and dopamine and the thyroid hormone thyroxine.

Variations in brain of L-tryptophan concentration modify the synthesis and release of serotonin (5-hydroxytryptamine), while variations in brain L-tyrosine concentration modify the synthesis and release of the catecholamine neurotransmitters (dopamine, norepinephrine and epinephrine).

Concentrations of amino acids in the brain is determined by the relative concentrations of amino acids crossing the blood-brain barrier (BBB) (Pardridge et al., 1975). Amino acids compete for active transport across this membrane which structurally similar amino acids. Tyrosine and tryptophan are both members of the large neutral amino acid (LNAA) category, which also includes phenyl-alanine, leucine, isoleucine, valine, histidine and methionine (Belitz et al., 2009).

LNAA enter into the brain via a transporter located on capillary endothelial cells in the locus of the blood-brain barrier which is saturable and competitive. Brain concentrations of either tryptophan or tyrosine are readily modified by their ingestion and by the ingestion of other LNAA that share a competitive transporter for uptake into brain from the circulation. The amount of tryptophan or tyrosine that actually enters the brain depends on the ratio of the amino acid in the plasma to the sum of the plasma concentrations of LNAA.

As a result, physiologic and pathophysiologic factors that influence blood concentrations of these LNAA and others that compete with them for a common transporter across the blood brain barrier predictably alter aromatic amino acid concentrations in brain, the formation and release of these monoamine transmitters, and consequently brain function (Fernstrom 1990, 1983). Treatments that influence the relative blood concentrations of L-tryptophan, L-tyrosine and the other large neutral amino acids can, therefore, also influence brain neurotransmitter synthesis.

Due to its role in the serotonin pathway, tyrosine and tryptophan levels supplements are associated with the medical treatment of different diseases such as depression, sleep disorders, cognitive disorders, anxiety, or neurodegenerative diseases.

International Patent Publication WO 2015188280 A1 (Meyer) discloses a method for treating or preventing postpartum blues in a subject in need thereof comprising the administration of an antioxidant source, a tryptophan composition comprising from 2 g of L-tryptophan, and a tyrosine composition comprising from 10 g of L-tyrosine, wherein the antioxidant source is for use at least once between day-1 to day-5 postpartum, tryptophan composition for evening administration simultaneously or following the antioxidant source between day-3 to day-5 postpartum and tyrosine composition is administered the day after administering the tryptophan composition. In the examples, the antioxidant source is administered by combining a beverage composition comprising blueberry concentrated juice and a sachet composition comprising blueberry extract. The tryptophan is administered in the form of 1 g tablets and tyrosine is administered as 0.5 mg capsules. Both the beverage composition and the sachet composition comprise sugar to render the treatment more palatable to the subject. Sugar amounts to 9.25% w/w of the antioxidant composition. Other sweeteners can be used if sugar is not desired. To comply with this dosage regimen, subjects are directed to drink concentrated blueberry juice combined with blueberry extract, and to take 2 capsules of 1 gram tryptophan each or 20 capsules of 0.5 mg tyrosine each, a dose which may involve clinical supervision.

Orally administrable liquid pharmaceutical compositions are attractive dosage forms for the administration of a prescribed dosage regimen. Liquid pharmaceutical compositions are easy to swallow, and if formulated appropriately, have an appealing taste, which may improve patient compliance with a prescribed dosing regimen. In addition, when compared to solid dosage forms, liquid pharmaceutical formulations provide better individualized dosing, which may be important when treating different patient populations.

Oral solutions comprising L-tyrosine or L-tryptophan known in the art typically contain less than 3% w/w carbohydrate (sugars) among its components. For example, patent publication WO 2004/047565 A1 (Tsunoo et al.) describes an amino-acid oral solution comprising tyrosine, citric acid, sucrose, trehalose and water for use as body temperature increasing agent. Carbohydrates present in the composition amount a 3% w/w of the total weight of the composition and are typically used as sweeteners or taste-masking agents.

Drugs administered in oral liquid form are immediately available for absorption from the gastrointestinal tract and can be absorbed faster than the same amount of drug administered in a tablet or capsule. However, in order to improve brain uptake of L-tyrosine and L-tryptophan, specially of L-tryptophan, it is not enough to increase the serum concentration of both amino acids. Co-administration of L-tryptophan or L-tyrosine with other LNAA leads to a depletion of the concentrations of these amino acids in brain. For further consideration of relative deficit in tryptophan or tyrosine relative to LNAA, reference is made to: Gessa et al. 1974; Young et al. 1985; and Le Masurier et al., 2006.

In spite of the efforts made there is still the need of oral liquid formulations with an effective uptake amino acids such as L-tryptophan or L-tyrosine.

### SUMMARY OF THE INVENTION

The inventors of present application have developed an oral liquid composition comprising an effective amount of an amino acid selected from the group of L-tryptophan and L-tyrosine which can obviate the problems associated with prior art and increases patient compliance, maximizing at the same time brain uptake of the amino acid present in the composition.

A first aspect of the invention is to provide an oral liquid composition comprising:
an effective amount of an amino acid selected from L-tyrosine or L-tryptophan,
at least one carbohydrate compound,
water, and
one or more acceptable excipients,
   wherein the composition comprises more than 5% w/w of the carbohydrate compound based on the total weight of the composition, and the weight ratio between the amino acid and the carbohydrate is from 1:1 to 1:50.

Without being bound to the theory, the present inventors believe that at low amounts, below the 5% w/w level, the role of the carbohydrate is as sweetener, but that when the amount is above 5% the same carbohydrate can help in facilitating the intake or absorption of the amino acid incorporated in the composition by promoting insulin response (Lee and Wolever, 1998). That is, the carbohydrate plays a role as adjuvant.

A second aspect of the invention is to provide an oral liquid composition comprising an effective amount of one amino acid selected from L-tyrosine and L-tryptophan, at least one carbohydrate compound, water, one or more pharmaceutically acceptable excipients for use in a method of treatment or prevention of postpartum blues or depression in a subject in need thereof, or for preparation of medicaments for treating or preventing postpartum blues or depression.

There is also provided herein a method of treating or preventing, and a corresponding use in treating or preventing, postpartum blues or depression comprising administering to a subject in need thereof an oral liquid composition as defined in the first or second aspect of the invention.

The present invention, in a final aspect also provides kits which include the oral liquid compositions of the invention of the methods and uses, together with instructions for use.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

For the purposes of the present invention, any ranges given include both the lower and the upper end-points of the range.

As used herein, the meaning of the term “comprising” encompasses three alternatives, namely “comprising”, “consisting of” and “consisting essentially of”.

As used herein, the term “oral liquid composition” refers to liquid compositions stored in physically discrete units suitable as unitary dosage which can be administered to a subject to provide the required amount of an active ingredient, such as L-tyrosine or L-tryptophan. With respect to the dosage form of the invention, the term “oral” refer to any method of administration through the mouth.

As used herein, the term “postpartum blues” are defined as low mood and mild depressive symptoms (including sadness, crying, exhaustion, irritability, anxiety, decreased sleep, decreased concentration), and labile mood that are transient and self-limited conditions that begin shortly after childbirth.

As used herein a “therapeutically effective amount” refers to an amount effective, at dosages and for periods of time necessary to achieve the desired therapeutic result, such as one or more of the following therapeutic results, such as a significant delay of the onset or progression of the disease; or a significant reduction of the severity of one or more symptoms. A therapeutically effective amount is also typically one in which any toxic or detrimental effect of the active ingredient or pharmaceutical composition is outweighed by the therapeutically beneficial effects.

The aim of the present invention was therefore to develop an oral liquid composition comprising an effective amount of an amino acid selected from the group consisting of L-tyrosine or L-tryptophan, at least one carbohydrate, water and optionally pharmaceutically acceptable excipients.

In one embodiment, the oral liquid composition according to the invention comprises an effective amount of L-tyrosine. Advantageously, the oral liquid composition comprises from 1.0 g to 50.0 g of L-tyrosine, preferably from 2.0 g to 40.0 g, more preferably from 3.0 g to 30.0 g, and even more preferably from 5 g to 20.0 g of L-tyrosine. In a most preferred embodiment, the oral liquid composition according to the invention comprises 5.0 g, 6.0 g, 7.0 g, 8.0 g, 9.0 g, 10.0 g, 11.0 g, 12.0 g, 13.0 g, 14.0 g, 15.0 g, 16.0 g, 17.0 g, 18.0 g, 19.0 g or 20.0 g of L-tyrosine. In the most preferred embodiment, the oral liquid composition according to the invention comprises 10.0 g L-tyrosine.

In a further embodiment, the oral liquid composition according to the invention comprises an effective amount of L-tryptophan. Advantageously, the oral liquid composition comprises from 0.5 g to 20.0 g of L-tryptophan, more preferably from 1 to 10 g, even more preferably from 1 to 5 g. The oral liquid composition according to the invention can comprise 1.0 g, 2.0, 3.0, 4.0 or 5.0 g L-tryptophan. In the most preferred embodiment, the composition according to the invention comprises 2 g of L-tryptophan.

In some specific embodiments of the present invention, the oral liquid composition according to the invention is substantially free from other amino acids selected from phenyl-alanine, leucine, isoleucine, valine, histidine and methionine to avoid partial depletion of tryptophan or tyrosine brain uptake from the composition of the invention.

In the context of the present invention, “substantially free” means that the composition comprises an amount of the above amino acids below 2% w/w, in particular below 1% w/w, in particular below 0.5% w/w.

In some embodiments, the oral liquid composition according to the invention comprises from about 2 mg/mL to 100 mg/mL of L-tyrosine, preferably from 3 mg/ml to 85 mg/mL, more preferably from 5 mg/mL to 75 mg/mL, and even more preferably from 6 mg/ml to 65 mg/ml of L-tyrosine. In the most preferred embodiment, the oral liquid composition according to the invention comprises from 45 mg/mL to 55 mg/ml of L-tyrosine.

In a further embodiment, the oral liquid composition according to the invention comprises from 2.5 mg/mL to 100 mg/mL of L-tryptophan, preferably from 3 mg/mL to 85 mg/mL, more preferably from 5 mg/mL to 75 mg/mL, and even more preferably from 6 mg/ml to 65 mg/mL g of L-tryptophan. In the most preferred embodiment, the oral liquid composition according to the invention comprises 8 mg/mL to 12 mg/ml of L-tryptophan.

In one embodiment, the oral liquid composition according to the invention comprises a carbohydrate at an amount of about 5% to about 50 percent w/w, or about 10% to about 40% w/w, or about 15% to about 30% w/w of the total weight of the composition. In some embodiments, the formulation comprises at least one carbohydrate at an amount of 20% to about 25% w/w of the total weight of the composition. In preferred embodiments, the carbohydrate is in the formulation at an amount higher than 5% w/w of the total weight of the composition, more preferably higher than 10% w/w of the total weight of the composition, more preferably higher than 15% w/w of the total amount of the composition.

In a further embodiment the oral liquid composition according to the invention comprises from 50 to 60 g carbohydrate, preferably from 53 to 57 g carbohydrate, wherein the carbohydrate is from 5%-30% w/w with respect to the total weight of the composition.

In some embodiments, the carbohydrate is a simple carbohydrate (sugar) or a saccharide. In a preferred embodiment, the saccharide is one or more of glucose, galactose, fructose, xylose, sucrose, lactose, maltose, mannose, trehalose, sorbitol, mannitol, xylitol, raffinose, cellobiose, inulin, malt polysaccharide, starch and cellulose or a combination thereof. Preferred saccharides are selected from sucrose, fructose and mixtures thereof.

In one embodiment, the oral liquid composition according to the invention comprises a carbohydrate selected from sucrose, fructose or mixtures thereof, at a concentration of about 5% to about 50 percent w/w, or about 10% to about 40% w/w, or about 15% to about 30% w/w of the total weight of the composition. In some embodiments, the formulation comprises at least one carbohydrate selected from sucrose, fructose or mixtures thereof, at a concentration of 20% to about 25% w/w of the total weight of the composition. In preferred embodiments, the carbohydrate is selected from sucrose, fructose or mixtures thereof and it is in the formulation at a concentration higher than 5% w/w of the total weight of the composition, such as higher than 10% w/w of the total weight of the composition, for example higher than 15% w/w of the total amount of the composition.

In some embodiments, the weight ratio of amino acid:carbohydrate is from 1:1 to 1:50. In preferred embodiments the weight ratio of amino acid:carbohydrate is from 1:2 to 1:40, more preferably form 1:3 to 1:30. In a most preferred embodiment the weight ratio of amino acid:carbohydrate is from 1:5 to 1:26.

In one embodiment, the composition comprises L-tryptophan and it is in a weight ratio with respect the carbohydrate from 1:10 to 1:40, from 1:20 to 1:35, or from 1:25 to 1:30.

In an alternative embodiment, the composition comprises L-tyrosine and it is in a weight ratio with respect the carbohydrate from 1:2 to 1:30, 1:3 to 1:15, from 1:4 to 1:10.

In the present invention the “weight ratio of amino acid:carbohydrate” is defined as the ratio between the amount of the amino acid vs the amount of carbohydrate, both amounts expressed in the same units (grams or milligrams).

The oral liquid composition according to the invention comprises water as solvent. In some embodiments, water is present in an amount of 50% to about 95% w/w, or about 60% to about 85% w/w, or about 65% to about 80% w/w of the total weight of the composition. In a preferred embodiment, the oral liquid composition according to the invention comprises about 68% to about 78% w/w of the total weight of the composition. In the most preferred embodiment, oral liquid composition according to the invention comprises from about 72% to about 78% w/w of the total weight of the composition.

In a preferred embodiment the liquid composition according to the invention comprises at least one ingredient having antioxidative properties.

The term “antioxidant” as used herein refers to the capacity of a substance included in the antioxidant composition, or may encompass a precursor compound which, upon consumption, is converted to an antioxidant within the body.

The ingredient having antioxidative properties according to the present invention can be of natural origin and is preferably obtained from a plant. Such an ingredient can be a plant extract or a plant powder. Plant includes here all parts of the plant such as leaves bark, seeds or fruits such as berries.

Preferred components of plant origin having antioxidant properties are selected from a group comprising polyphenols, in particular plant polyphenols such as flavonoids, ellagitannins, xanthones, tannins and anthocyanins. In a most preferred embodiment, the ingredient having antioxidative properties and/or anti-oxidant enzyme inducing properties are anthocyanins. Anthocyanins may be isolated from blueberries and can be administered as blueberry extract.

In some embodiments, the ingredient having antioxidative properties can be selected Vitamin C, Vitamin E, indole-3-carbinol, glutathione or mixtures thereof.

In one embodiment, the composition is a food supplement. As used herein the term “food supplement”, also so-called “nutritional supplement”, refers to concentrated sources of nutrients or other substances with a nutritional or physiological effect whose purpose is to supplement the normal diet. In other terms food supplement means any food the purpose of which is to supplement the normal diet and which is a concentrated source of a vitamin or mineral or other substance with a nutritional or physiological effect, alone or in combination. A food supplement is preferably sold in dose form. The definition of “to supplement” can be interpreted as taken in addition to the diet.

In another embodiment, the composition is a pharmaceutical or nutraceutical composition.

The oral liquid composition may optionally include at least one taste-masking agent or bitter blocker agent. Taste-masking agents include sweetening agents and flavoring agents, which may be used alone or in combination.

In addition to the sweetening agent, the liquid pharmaceutical or nutraceutical composition may include a flavoring agent. Useful flavoring agents include various fruit flavors (e. g. orange, cherry, strawberry, grape, blueberry, etc.), mint, cocoa, chocolate and vanilla flavor.

The oral liquid pharmaceutical or nutraceutical composition may optionally include a viscosity control agent to raise the composition's viscosity. Increasing the viscosity improves the handling of the composition and appears to improve the taste of the pharmaceutical or nutraceutical preparation. Useful viscosity control agents include hydroxyethyl cellulose, xanthan gum, guar gum, and the like, which may be used separately or in combination. Preferred viscosity control agent according to the invention is guar gum.

The viscosity control agent may be used at a concentration of at least about 0.001 mg/ml up to a concentration of about 5 mg/mL.

There is also provided herein a method of treating or preventing, and a corresponding use in treating or prophylaxis/prevention of postpartum blues or depression comprising administering to a subject in need thereof an oral liquid composition as defined in any of the above aspects and embodiments.

As used herein, “treatment” refers to addressing postpartum blues or depressed mood arising in the postpartum period in an individual already experiencing symptoms of and tendencies toward postpartum blues or depressed mood in a manner that results in improvement. Improvement in postpartum blues or sad mood may be referenced herein interchangeably with improvement in postpartum blues or depressed mood. Treatment need not entirely cure or eradicate the blues or depressed mood of the individual, but may simply lessen the strength or duration one or more of the symptoms such as dysfunctional attitude, crying, irritability, poor concentration, appetite alterations, tiredness, depression, anxiety, change in calmness or tension, restlessness, confusion, anger, and/or fatigue. Improvement in mood may be determined by the individual, by a health care professional, or may be determined using accepted parameters such as surveys, questionnaires, or physiological tests. Amelioration of symptoms without entirely eradicating postpartum blues or depressed mood is considered as treatment. Treatment may be a reduction in severity or duration of symptoms as determined by self-reporting, or clinically accepted parameters used to assess postpartum blues or depressed mood, including questionnaires or behavior evaluation techniques.

As used herein, “prevention” or “prophylaxis” of depressed mood arising in the postpartum period means addressing depressed mood prior to its occurrence in a manner that prevents, delays, or lessens the occurrence entirely or in part. Prevention or prophylaxis also encompasses addressing depressed mood in advance of occurrence or in advance of severe onset in a manner that lessens the severity, duration, or likelihood of recurrence of the depressed mood and its symptoms, resulting in an improvement for the individual. “Prevention” and “prophylaxis” do not mean that depressed mood will not occur in the individual, but indicates that measures are taken before depressed mood onset in an individual in order to address the problem in advance. There is benefit to the individual to having a preventative or prophylactic strategy in place prior to onset, which can be utilized proactively even if depressed mood can only be prevented in part. Methods and compositions for addressing depressed mood are described in WO 2015/1188280 A1 (Meyer).

In one embodiment, the oral liquid composition of the invention is administered to the subject on day-3 to day-5 postpartum.

In another embodiment, the oral liquid composition of the invention is administered once, twice or three times a day. In another embodiment, the oral liquid composition of the invention is administered once a day.

In another embodiment, the method comprises administering an oral liquid composition of the invention comprising an effective amount of L-tyrosine that is effective in averting or reducing symptoms, and/or that is therapeutically effective. In an alternative embodiment, the method comprises administering an oral liquid composition of the invention comprising an effective amount of L-tryptophan that is effective in averting or reducing symptoms, and/or that is therapeutically effective. In an alternative embodiment, the method comprises administering, either simultaneously, sequentially or separately an oral liquid composition of the invention comprising a therapeutically effective amount of L-tyrosine and an oral liquid composition comprising a therapeutically effective amount of L-tryptophan. In an alternative embodiment, the method comprises administering sequentially an oral liquid composition of the invention comprising a therapeutically effective amount of L-tyrosine and an oral liquid composition of the invention comprising a therapeutically effective amount of L-tryptophan. In an alternative embodiment, the method comprises administering sequentially and in different days, an oral liquid composition of the invention comprising a therapeutically effective amount of L-tyrosine and an oral liquid composition of the invention comprising a therapeutically effective amount of L-tryptophan. In an alternative embodiment, the method comprises administering firstly an oral liquid composition of the invention comprising a therapeutically effective amount of L-tryptophan and subsequently the oral liquid composition of the invention comprising a therapeutically effective amount of L-tyrosine. In an alternative embodiment, the method comprises administering sequentially and in different days, firstly the oral liquid composition of the invention comprising a therapeutically effective amount of L-tryptophan and subsequently the oral liquid composition of the invention comprising a therapeutically effective amount of L-tyrosine. In an alternative embodiment, the method comprises administering firstly the oral liquid composition of the invention comprising a therapeutically effective amount of L-tryptophan 0 and the oral liquid composition of the invention comprising a therapeutically effective amount of L-tyrosine the following day. In an alternative embodiment, the L-tryptophan and the L-tyrosine solutions are administered between day-1 to day-5 postpartum. In an alternative embodiment, it is firstly administered the L-tryptophan solution and subsequently L-tyrosine solution, between day-1 to day-5 postpartum. In an alternative embodiment, it is firstly administered the L-tryptophan solution and in a subsequent different day the L-tyrosine solution, between day-1 to day-5 postpartum.

In an alternative embodiment, the tryptophan composition is firstly administered on day-4 postpartum, and the tyrosine oral liquid composition is administered on day-5 postpartum.

The method of the invention can include the administration of at least a further antioxidant composition, either in the form of a liquid or solid form, substantially free of amino acids. In one embodiment, the further antioxidant composition is separately administered. In one embodiment, the further antioxidant composition, either in solid or liquid form, is administered before administering the tryptophan composition. In another embodiment, the further antioxidant composition, either in liquid or solid form, is administered on day 3-postpartum. In another embodiment, the antioxidant composition, either in liquid or solid form, is administered on day-3 and day-4 postpartum, the tryptophan composition on day-4 post-partum and the tyrosine composition on day-5 postpartum.

According to a further aspect, the oral liquid composition according to the invention can be used as part of a multi-component kit. Such kit, which is suitable for use in treating or preventing postpartum blues or depression comprises:
a. an oral liquid composition comprising an effective amount L-tryptophan, at least one carbohydrate compound at a % by weight of at least 5% w/w, the weight ratio between the amino acid and the carbohydrate is from 1:1 to 1:50, water, and optionally pharmaceutically acceptable excipients;
b. an oral liquid composition comprising an effective amount L-tyrosine, at least one carbohydrate compound at a % by weight of at least 5% w/w, the weight ratio between the amino acid and the carbohydrate is from 1:1 to 1:50, water, and optionally pharmaceutically acceptable excipients; and
   instructions for the administration of both compositions, for example, for administration between day-4 to day-5 postpartum.

Typically, the kit further comprises an oral liquid beverage comprising at least one carbohydrate compound, water at least one ingredient having antioxidant properties and optionally one or more acceptable excipients is administered on days 1 to 3 postpartum.

According to a preferred embodiment the kit comprises:
a. an oral liquid composition comprising an effective amount L-tryptophan, at least one carbohydrate compound at a % by weight of at least 5% w/w, the weight ratio between the amino acid and the carbohydrate is from 1:1 to 1:50, water, at least one ingredient having antioxidant properties and optionally one or more acceptable excipients;
b. an oral liquid composition comprising an effective amount L-tyrosine, at least one carbohydrate compound at a % by weight of at least 5% w/w, the weight ratio between the amino acid and the carbohydrate is from 1:1 to 1:50, water, at least one ingredient having antioxidant properties and optionally one or more acceptable excipients;
c. oral composition, either in liquid or solid form, comprising at least one carbohydrate compound, water at least one ingredient having antioxidant properties and optionally one or more acceptable excipients, wherein the oral composition is substantially free of amino acids; and
   instructions for the administration, for example for administration between day-1 to day-5 postpartum.

The oral solid composition comprising the antioxidant ingredient can take the form of a pill, tablet, powder or capsule, for instance, of any antioxidant suitable in the context of the invention (see above). There are well-known and commercially available antioxidant solid compositions.

The kit of the invention provides instructions for the administration of the oral liquid compositions of the invention as provided in any of the above embodiments related to the administration of the formulations (separately, sequentially, simultaneously; at different or the same say; in the particular case of post-partum blues, the particular dosage regimens, etc.). Therefore, the above embodiments provided under the method of treatment and prevention are also embodiments when using the kit of the invention.

According to a further aspect, the kit comprises a plurality of liquid compositions together with instructions for oral administration in a multi-day postpartum regime for treatment or prophylaxis of postpartum blues or depressed mood in a subject in need thereof.

The plurality of liquid compositions comprise:
a first composition and a second composition comprising 50-60 g carbohydrate and an antioxidant source, wherein the carbohydrate is from 5%-30% w/w with respect to the total liquid weight of the composition, and wherein the first and second composition are essentially free of amino acids and may be the same or different from each other;
a third composition comprising 50-60 g carbohydrate, an antioxidant source, and an amino acid consisting of L-tryptophan, wherein the weight ratio of L-tryptophan to carbohydrate is from 1:10 to 1:40, and the carbohydrate is from 5%-30% w/w with respect to the total liquid weight of the composition; and
a fourth composition comprising 50-60 g carbohydrate, an antioxidant source, and an amino acid consisting of L-tyrosine, wherein the weight ratio of L-tyrosine to carbohydrate is from 1:2 to 1:30, and the carbohydrate is from 5%-30% w/w with respect to the total liquid weight of composition; and
instructions for oral administration in a multi-day postpartum regime for treatment or prophylaxis of postpartum blues or depressed mood in a subject in need thereof.

For example, the instructions for use may direct administration of: the first composition on the evening of the first day of the postpartum regime; the second composition on the morning of the second day of the postpartum regime; the third composition on the evening of the second day of the postpartum regime; and the fourth composition on the morning of the third day of the postpartum regime.

For example, the third composition may comprise a weight ratio of L-tryptophan to carbohydrate of from 1:20 to 1:35 or from 1:25 to 1:30; and the fourth composition may comprise a weight ratio of L-tyrosine to carbohydrate of from 1:3 to 1:15 or from 1:4 to 1:10.

The kit may also have one or more of the following features: the first day of the regime may correspond with the subject's postpartum day 3; the antioxidant source may comprise blueberry extract and/or blueberry juice; the carbohydrate may be from 20%-30% w/w of the total weight of the composition; the third composition may comprises from 1-5 g of L-tryptophan, for example 2 g L-tryptophan; the fourth composition may comprise from 5-20 g of L-tyrosine, for example 10 g L-tyrosine; one or more of the compositions may be provided in the form of a shake; the carbohydrate may comprise a simple carbohydrate, for example sucrose; the antioxidant source may comprise a blueberry extract and/or juice providing 6500 ORAC units/g; and/or the compositions may further comprise an excipient or vehicle selected the group consisting of taste-masking agents, bitter blocker agents, sweeteners, flavorings, viscosity control agents, thickeners, guar gum, fiber, cocoa powder and combinations thereof.

The following examples are intended to be illustrative and non-limiting, and represent specific embodiments of the present invention.

### Example 1

### L-Tryptophan Oral Solution

The following oral solution, outlined in Table 1, is an example of an oral amino acid solution in which the amino acid L-tryptophan is present with a ratio of carbohydrate (primarily sucrose) of about 2 g: 55.84 g (or about 1:27.92). Further, the carbohydrate from sucrose, when expressed as a percent by weight of the total weight of the composition is 21.38%. The single serving represented in Table 1 weighs 261.274 g, and is to be consumed all at once with the sugar content of 55.84 g per serving, and thus has a high sugar content to help with absorption and uptake of the amino acid content. Excipients are present in this exemplary oral solution, and blueberry extract contributes antioxidant capacity to the solution. The solution can be delivered in the form of a prepared solution that can be shaken to enhance mixing and mouthfeel.

TABLE 1
Exemplary L-Tryptophan Oral Solution

| Material Description | Total Dose (g) | % of Formula |
|---|---|---|
| Water | 195.000 | 74.64% |
| Blueberry Extract 12% anthocyanins** | 4.004 | 1.53% |
| Sucrose | 55.840 | 21.38% |
| L-Tryptophan | 2.000 | 0.76% |
| Guar Gum (Fiber) | 0.030 | 0.01% |
| Natural Chocolate WONF Flavor | 0.800 | 0.31% |
| Cocoa Powder (Dutched) | 2.000 | 0.76% |
| Natural Vanilla Custard Flavor | 1.200 | 0.46% |
| Bitter Blocker-MTP00634 | 0.360 | 0.14% |
| Non Dairy Creamer Flavor | 0.040 | 0.01% |
| TOTAL | 261.274 | 100.000% |

**Blueberry extract provides 6500 ORAC units/g

### Example 2

### L-Tyrosine Oral Solution

The following oral solution in Table 2 is an example of an oral amino acid solution in which the amino acid L-tyrosine is present with a ratio of carbohydrate (primarily sucrose) of about 10.204 g: 55.84 g (or about 1:5.45). Further, the carbohydrate from sucrose, when expressed as a percent by weight of the total weight of the composition is 20.78%. The single serving represented in Table 2 weighs 268.678 g, and is to be consumed all at once with the sugar content of 55.84 g per serving, and thus has a high sugar content to help with absorption and uptake of the amino acid content. Excipients are present in this exemplary oral solution, and blueberry extract contributes antioxidant capacity to the solution. The solution can be delivered in the form of a prepared solution that can be shaken to enhance mixing and mouthfeel.

TABLE 2
Exemplary L-Tyrosine Oral Solution

| Material Description | Total Dose (g) | % of Formula |
|---|---|---|
| Water | 195.000 | 72.5% |
| Blueberry Extract 12% anthocyanins** | 4.004 | 1.49% |
| Sucrose | 55.840 | 20.78% |
| L-Tyrosine | 10.204 | 3.80% |
| Guar Gum (Fiber) | 0.030 | 0.01% |
| Natural Chocolate WONF Flavor | 0.800 | 0.30% |
| Cocoa Powder (Dutched) | 2.000 | 0.74% |
| Natural Vanilla Custard Flavor | 0.800 | 0.30% |
| TOTAL | 268.678 | 100.00% |

**Blueberry extract provides 6500 ORAC units/g

### Example 3 (Reference)

### Beverage Comprising an Antioxidant

The following oral solution, outlined in Table 3, is an example of a reference oral amino acid solution without amino acids. The carbohydrate present (primarily sucrose) is about 55.84 g. The carbohydrate from sucrose, when expressed as a percent by weight of the total weight of the composition is 21.35%. The single serving represented in Table 3 weighs 261.474 g, and is to be consumed all at once as a serving. Excipients are present in this exemplary oral solution, and blueberry extract contributes antioxidant capacity to the solution. The solution can be delivered in the form of a prepared solution that can be shaken to enhance mixing and mouthfeel.

TABLE 3

| Material Description | Total Dose (g) | % of Formula |
|---|---|---|
| Water | 200.000 | 76.49% |
| Blueberry Extract 12% anthocyanins** | 4.004 | 1.53% |
| Sucrose | 55.840 | 21.35% |
| Guar Gum (Fiber) | 0.030 | 0.01% |
| Natural Chocolate WONF Flavor | 0.800 | 0.31% |
| Natural Vanilla Custard Flavor | 0.800 | 0.31% |
| TOTAL | 261.474 | 100.00% |

**Blueberry extract provides 6500 ORAC units/g

### Example 4

### Composition for Postpartum Subjects

To complete the study pregnant women who are healthy and have no history of major depression episode (MDE) are recruited during their third trimester. Subjects will randomly be assigned to placebo or active supplement in a double blind manner within blocks of 2, 4, 6, 8 or 10 subjects. The supplements/placebo are taken at 4 time points, starting on day 3 postpartum night to day 5 postpartum morning (See below full schedule). Subjects receive either the liquid formulation or a placebo that matches in taste and color.

### Timing of Administration of Study Products:

Night of day 3 postpartum: Participants drink the antioxidant liquid composition as disclosed in Example 3 or placebo liquid composition.
Morning of day 4 postpartum Participants drink the antioxidant liquid composition as disclosed in Example 3 or placebo liquid composition.
Night of day 4 postpartum: Participants drink a tryptophan liquid composition as disclosed in Example 1 or a placebo liquid composition.
Morning of day 5 postpartum: Participants drink a tyrosine liquid composition as disclosed in Example 2 or a placebo liquid composition.

On day 5, after the administration of the liquid composition, participants are asked to complete the following instruments:
Pittsburgh Sleep Quality Index. This questionnaire is used to assess the sleep quality of the subjects.
First, subjects go through a neutral mood induction procedure (MIP) based on the Velten MIP, which is the most widely used technique for studying affective influences upon behavior and it has demonstrated effectiveness in altering subjective emotional states (Frost et al., 1982).

Following the neutral MIP, subjects will fill out the following questionnaires in the stated order:
a. Visual Analog Scale (VAS) uses a 10-point scale for participants to indicate the extent to which each of the 8 items is consistent with how they feel in the moment; the items included depressed, happy, restless, sad, anxious, angry, drowsy and alert. Within VAS, mood assessment will be done first.
b. Dysfunctional Attitude Scale (DAS) designed to identify and measure cognitive distortions, particularly distortions that may relate to or cause depression
c. Profile of Mood State (POMS) contains of 65 adjectives rated by participants on a 5-point scale. Six factors are derived that include tension, depression, anger, fatigue, vigor and confusion.
d. VAS
e. State-Trait Anxiety Inventory consists of two scales containing 20 items each. One scale addresses state anxiety while the other scale addresses trait anxiety. The total score indicates which type of anxiety is prevalent and distinguishes between a person's state and trait anxiety levels.
Second, subjects go through a sad mood induction, based on the Velten MIP. Following the sad MIP, subjects repeat above questionnaires in the stated order.
After the completion of the above questionnaires, subjects go through an Emotional Stroop Test. This test is used as an information-processing approach to assess emotions. The Emotional Stroop test works by examining the response time of the participant to name colors of negative emotional words.
VAS
Subjects will go through a neutral mood induction
VAS
Beck Depression Inventor Scale (BDI), standardized health questionnaire
Edinburgh Postnatal Depression Scale (EPDS), standardized health questionnaire
Stein Blue Scale is a self-rated scale and consists of 13 symptoms (depression, crying, anxiety, tension, restlessness, exhaustion, dreaming, appetite, headache, irritability, poor concentration, forgetfulness and confusion).
Kennerley and Gath is a self-rated 28 items blues questionnaire.
HAM-D, standardized health questionnaire
Center for Epidemiological Studies Depression Scale (CES-D) 165, is a 20-item depression inventory scored from 0 (least depressed) to 60 (most depressed).
Safety and tolerability checklist
Record for diet on day 4 postpartum night and 5 postpartum morning

Primary analysis uses of repeated measures analysis of variance with the visual analogue scale of depressed mood as the repeated measure (before and after the sad MIP) and active condition versus placebo as the between subject measure.

Secondary analysis is conducted on the change in depression scores on profile of mood state (POMS) scale, using repeated measures analysis of variance with the POMS score as the repeated measure (before and after the sad MIP) and active condition versus placebo as the between subject measure.

Methods and timing for assessing, recording, and analyzing safety parameters are as follows. Adverse events are recorded on an Adverse Event Log. The Principal Investigator is consulted to make a determination on the reasonable causal relationship to the investigational product and the seriousness of the event. Adverse events are collected until the end of the Day 5 post-partum study.

### Example 5

### Prepared Shakes for Treatment or Prevention of Postpartum Blues or Depressed Mood

A kit comprising multiple liquid servings is packaged together for use in a multi-day regimen by postpartum individuals experiencing or wishing to prophylactically avoid postpartum blues or depressed mood.

The kit may, for example, come with four sealed containers, each labelled for the time of day on the multi-day regimen to be consumed, for example as morning or evening of Day 1 (corresponding with day-3 postpartum), Day 2 (corresponding with day-4 postpartum), or Day 3 (corresponding with day-5 postpartum) of the regimen. The convenience of prepared liquid format in a sealed container permits the user to refrigerate and to shake the container well before use, which can mobilize stabilizers and other excipients such as thickeners for a thick and consistent mouthfeel. The convenience of a prepared product to be consumed on schedule in the hectic and disorienting postpartum period encourages easy compliance as parents adapt to myriad aspects of their situations, so shortly after childbirth. The clear labelling of each container and easy-to-follow instructions of the kit also promote compliance.

The sugar content of each individual container (representing a serving) may range from 40 to 65, for example from 55 g to 58 g per serving. Tyrosine or tryptophan content of the individual servings may be, for example: 2 g of L-tryptophan (as in Example 1, Table 1), or 10 g of L-tyrosine (as in Example 2, Table 2).

Each container may be sized consistently with the other containers in the kit, and may range from 250 to 300 mL, representing a reasonable volume for an individual to consumed all at once.

Thickeners and other ingredients to create a desirable mouthfeel representing a “shake” make the product more desirable to the subject.

Preparing the product in a convenient format, with easy to following packaging in a kit format, while ensuring the formulation is refreshing, delicious, fruity and healthful, encourages subject compliance. For example, 4 different but clearly labelled bottles may be sold in a pre-packaged box with easy-to-follow instructions, to be optionally refrigerated and shaken before opening. The instructions for use may follow, for example, the regimen outlined by Meyer (2015) in WO 2015188280 A1.

A sample regimen may be as follows:

TABLE 4
Sample Regimen and Kit Components

| Kit Component, | Approximate | |
|---|---|---|
| Postpartum Day | Time of Day | Components of Shake to be Consumed |
| Kit composition 1. | Evening | Shake contains ~50-60 g carbohydrate in a 250- |
| Day-3 postpartum | (for example | 300 mL volume serving with ANTIOXIDANT |
| (evening) | 9:00 pm) | SOURCE (blueberry extract) |
| | | [See Table 3] |
| Kit composition 2. | Morning | Shake contains ~50-60 g carbohydrate in a 250- |
| Day-4 postpartum | (for example | 300 mL volume serving with ANTIOXIDANT |
| (morning) | 10 am) | SOURCE (blueberry extract) |
| | | [See Table 3] |
| Kit composition 3. | Evening | Shake contains ~50-60 g carbohydrate in a 250- |
| Day-4 postpartum | (for example | 300 mL volume serving, with TRYPTOPHAN |
| | 9:00 pm) | (about 2 g L-tryptophan); and ANTIOXIDANT |
| | | SOURCE (blueberry extract) |
| | | [See Table 1] |
| Kit composition 4. | Morning | Shake contains ~50-60 g carbohydrate in a 250- |
| Day-5 postpartum | (for example | 300 mL volume serving, with TYROSINE (about |
| (morning) | 10 am) | 10 g L-tyrosine); and ANTIOXIDANT SOURCE |
| | | (blueberry extract) |
| | | [see Table 2] |

### REFERENCES

The following documents are herein incorporated by reference.
Belitz H D et al. Food Chemistry, 4^{th} revised and extended edition, 2009, Springer-Verlag Berlin Heidelberg. Chapter 1 Amino Acids, Peptides, Proteins. pages 8-92.
Fernstrom et al. (1983). Role Of Precursor Availability In The Control Of Monoamine Biosynthesis In Brain. Physiol. Rev 1983, 63, 484-546.
Fernstrom et al. (1990). Aromatic amino acids and monoamine synthesis in the central nervous system: influence of the diet. J. Nutr. Biochem. 1990, 1, 508-17.
Frost et al. (1982) Mood Induction Procedure Effects: Duration and Post-experimental Removal. Personality and Social Psychology Bulletin 1982, 8, 341-347.
Gessa et al. (1974) Effect of the oral administration of tryptophan-free amino acid mixtures on serum tryptophan, brain tryptophan, and serotonin metabolism. J. Neurochem 1974, 22, 869-870.
Lee and Wolever (1998) Effect of Glucose, Sucrose and Fructose on Plasma Glucose and Insulin Responses in Normal Humans: Comparison with White Bread. European Journal of Clinical Nutrition 1998, 52, 924-928.
Le Masurier et al. (2006) Effect of Acute Tyrosine Depletion in Using a Branched Chain Amino-Acid Mixture on Dopamine Neurotransmission in the Rat Brain. Neuropsychopharmacology 2006, 31, 310-317.
Meyer (2015) Methods and Compositions Addressing Depressed Mood. International Patent Application PCT/CA2015/050548 filed Jun. 12, 2015 published as WO 2015188280 A1 on Dec. 17, 2015.
Pardridge et al. (1975). Kinetic Analysis Of Blood Brain Barrier Transport Of Amino Acids. Biochim Biophys. Acta 1975, 401, 128-136.
Young et al. (1985) Biochemical aspects of tryptophan depletion in primates. Psychopharmacology 1985, 98, 508-511.
Tsunoo et al. (2004) Agent for Foods, Drinks or Medical Uses Containing Body Temperature-Elevating Amino Acid. WO 2004/047565 A1 published Jun. 10, 2004.

### Clauses

For reasons of completeness, various aspects of the invention are set out in the following numbered clauses:

Clause 1. An oral liquid composition comprising an effective amount of an amino acid selected from L-tyrosine and L-tryptophan; a carbohydrate, water, and optionally one or more acceptable excipients, wherein the carbohydrate is at a % by weight of at least 5% w/w with respect to the total weight of composition, and the weight ratio between the amino acid and the carbohydrate is from 1:1 to 1:50.

Clause 2. The oral liquid composition according to clause 1, wherein:
the amino acid is L-tyrosine, and the carbohydrate is at a weight ratio excess with respect the amino acid, particularly the weight ratio between the L-tyrosine and the carbohydrate is from 1:2 to 1:30, 1:3 to 1:15, or from 1:4 to 10; or, alternatively,
the amino acid is L-tryptophan, and the carbohydrate is at a weight ratio excess with respect the amino acid, particularly the weight ratio between the L-tryptophan and the carbohydrate is 1:10 to 1:40, from 1:20 to 1:35, or from 1:25 to 1:30.

Clause 3. The oral liquid composition according to clause 1 or 2, wherein the carbohydrate is at a % w/w from about 5% to about 50 percent w/w, or about 10% to about 40% w/w, or about 15% to about 30% w/w of the total weight of the composition.

Clause 4. The oral liquid composition according to any one of clauses 1 to 3, wherein the oral liquid composition comprises from 5 g to 20.0 g of L-tyrosine, particularly 10 g L-tyrosine.

Clause 5. The oral liquid composition according to any one of clauses 1 to 4, wherein the oral liquid composition comprises from 1 to 10 g L-tryptophan, particularly 2 g of L-tryptophan.

Clause 6. The oral liquid composition according to any one of clauses 1 to 5, wherein the composition comprises from 3 mg/mL to 75 mg/mL of L-tyrosine or L-tryptophan.

Clause 7. The oral liquid composition according to any one of clauses 1 to 6, further comprising at least one ingredient having antioxidant properties.

Clause 8. The oral liquid composition according to clause 7, wherein the ingredient having antioxidant properties is selected from the group consisting of flavonoids, ellagitannins, xanthones, tannins, anthocyanins, Vitamin C, Vitamin E, indole-3-carbinol, glutathione, and mixtures thereof.

Clause 9. The oral liquid composition according to any one of clauses 1 to 8, wherein the composition is substantially free from other amino acids selected from phenyl-alanine, leucine, isoleucine, valine, histidine and methionine.

Clause 10. The oral liquid composition according to any one of clauses 1 to 9, wherein the composition comprises one or more suitable excipients or vehicles selected from taste-masking agent, bitter blocker agent, sweetener agent, flavoring agent and viscosity control agents.

Clause 11. The composition according to any one of clauses 1 to 10, which is a food supplement and comprises edible acceptable excipients.

Clause 12. The composition according to any one of clauses 1 to 10, which is a pharmaceutical composition and comprises pharmaceutically acceptable excipients.

Clause 13. A kit:
a. an oral liquid composition as defined in any one of clauses 1 to 12, wherein the amino acid is L-tryptophan;
b. an oral liquid composition comprising as defined in any one of clauses 1 to 12, wherein the amino acid is L-tyrosine;
and
instructions for the administration of both compositions.

Clause 14. A kit according to clause 13 comprising:
a. an oral liquid composition as defined in any one of clauses 1-12, wherein the amino acid is L-tryptophan;
b. an oral liquid composition comprising as defined in any one of clauses 1-12, wherein the amino acid is L-tyrosine;
c. an oral composition, either liquid or solid, comprising at least one carbohydrate compound, water at least one ingredient having antioxidant properties and optionally one or more acceptable excipients, wherein the oral liquid solution is substantially free of amino acids; and
   instructions for the administration.

Clause 15. A method for the treatment or prevention of postpartum blues or depression in a subject in need thereof, the method comprising administering the oral liquid composition as defined in any one of clauses 1-12, comprising a therapeutically effective amount of one amino acid selected from L-tyrosine and L-tryptophan, the at least one carbohydrate compound, water and a pharmaceutically acceptable excipients or, alternatively, using the kit as defined in clause 13 or 14.

Clause 16. The method according to clause 15, wherein the tryptophan oral liquid composition and the tyrosine oral liquid formulations are not administered on the same day.

Clause 17. The method according to clause 15 or 16, wherein the tryptophan oral liquid composition is administered on day-4 postpartum and the tyrosine oral liquid composition is administered on day-5 postpartum.

Clause 18. The method according to any one of clauses 15 to 17, which further comprises administering an oral liquid composition comprising an antioxidant compound as defined above.

Clause 19. The method according to clause 18, wherein the further antioxidant compound is administered day-3 postpartum.

Clause 20. The method according to clause 19, wherein the further antioxidant compound is further administered day 4-postpartum.

Clause 21. A kit comprising a plurality of liquid compositions together with instructions for oral administration in a multi-day postpartum regime for treatment or prophylaxis of postpartum blues or depressed mood in a subject in need thereof, said plurality of liquid compositions comprising:
a first composition and a second composition comprising 50-60 g carbohydrate and an antioxidant source, wherein the carbohydrate is from 5%-30% w/w with respect to the total liquid weight of the composition, wherein the first and second composition are essentially free of amino acids and may be the same or different from each other;
a third composition comprising 50-60 g carbohydrate, an antioxidant source, and an amino acid consisting of L-tryptophan, wherein: the weight ratio of L-tryptophan to carbohydrate is from 1:10 to 1:40, and the carbohydrate is from 5%-30% w/w with respect to the total liquid weight of the composition; and
a fourth composition comprising 50-60 g carbohydrate, an antioxidant source, and an amino acid consisting of L-tyrosine, wherein: the weight ratio of L-tyrosine to carbohydrate is from 1:2 to 1:30, and the carbohydrate is from 5%-30% w/w with respect to the total liquid weight of composition; and
instructions for administration of:
the first composition on the evening of the first day of the postpartum regime;
the second composition on the morning of the second day of the postpartum regime;
the third composition on the evening of the second day of the postpartum regime; and
the fourth composition on the morning of the third day of the postpartum regime.

Clause 22. The kit of clause 21, wherein:
the third composition comprises a weight ratio of L-tryptophan to carbohydrate of from 1:20 to 1:35 or from 1:25 to 1:30; and
the fourth composition comprises a weight ratio of L-tyrosine to carbohydrate of from 1:3 to 1:15 or from 1:4 to 1:10.

Clause 23. The kit of clause 21 or 22, wherein:
the first day of the regime corresponds with the subject's postpartum day 3;
the antioxidant source comprises blueberry extract and/or blueberry juice;
the carbohydrate is from 20%-30% w/w of the total weight of the composition;
the third composition comprises from 1-5 g of L-tryptophan, or 2 g L-tryptophan;
one or more of the compositions is in provided in the form of a shake;
the carbohydrate comprises simple carbohydrates, or sucrose;
the fourth composition comprises from 5-20 g of L-tyrosine, or 10 g L-tyrosine;
the antioxidant source comprises a blueberry extract and/or juice providing 6500 ORAC units/g; and/or
the compositions further comprise one or more excipient or vehicle selected the group consisting of taste-masking agents, bitter blocker agents, sweeteners, flavorings, viscosity control agents, thickeners, guar gum, fiber, and/or cocoa powder.

1. An oral liquid composition comprising an effective amount of an amino acid selected from L-tyrosine and L-tryptophan; a carbohydrate, water, and optionally one or more acceptable excipients, wherein the carbohydrate is at a % by weight of at least 5% w/w with respect to the total weight of composition, and the weight ratio between the amino acid and the carbohydrate is from 1:1 to 1:50.

2. The oral liquid composition according to claim 1, wherein:
the amino acid is L-tyrosine, and the carbohydrate is at a weight ratio excess with respect the amino acid, particularly the weight ratio between the L-tyrosine and the carbohydrate is from 1:2 to 1:30, 1:3 to 1:15, or from 1:4 to 1:10; or, alternatively,
the amino acid is L-tryptophan, and the carbohydrate is at a weight ratio excess with respect the amino acid, particularly the weight ratio between the L-tryptophan and the carbohydrate is 1:10 to 1:40, from 1:20 to 1:35, or from 1:25 to 1:30.

3. The oral liquid composition according to claim 1, wherein the carbohydrate is at a % w/w from about 5% to about 50 percent w/w, or about 10% to about 40% w/w, or about 15% to about 30% w/w of the total weight of the composition.

4. The oral liquid composition according to claim 1, wherein the composition comprises from 3 mg/mL to 75 mg/mL of L-tyrosine or L-tryptophan.

5. The oral liquid composition according to claim 1, further comprising at least one ingredient having antioxidant properties.

6. (canceled)

7. The oral liquid composition according to claim 1, wherein the composition comprises one or more suitable excipients or vehicles selected from the group consisting of taste-masking agent, bitter blocker agent, sweetener agent, flavoring agent and viscosity control agents.

8. The composition of claim 1, which is a food supplement.

9. The composition of claim 1, which is a pharmaceutical composition and comprises one or more pharmaceutically acceptable excipients.

10. A kit comprising:
a. an oral liquid composition comprising an effective amount L-tryptophan, at least one carbohydrate compound at a % by weight of at least 5% w/w, the weight ratio between the amino acid and the carbohydrate is from 1:1 to 1:50, water, and optionally one or more pharmaceutically acceptable excipients;
b. an oral liquid composition comprising an effective amount L-tyrosine, at least one carbohydrate compound at a % by weight of at least 5% w/w, the weight ratio between the amino acid and the carbohydrate is from 1:1 to 1:50, water, and optionally one or more pharmaceutically acceptable excipients; and
instructions for the administration of both compositions.

11. A kit comprising:
a. an oral liquid composition comprising an effective amount L-tryptophan, at least one carbohydrate compound at a % by weight of at least 5% w/w, the weight ratio between the amino acid and the carbohydrate being from 1:1 to 1:50, water, at least one ingredient having antioxidant properties and optionally one or more acceptable excipients;
b. an oral liquid composition comprising an effective amount L-tyrosine, at least one carbohydrate compound at a % by weight of at least 5% w/w, the weight ratio between the amino acid and the carbohydrate being from 1:1 to 1:50, water, at least one ingredient having antioxidant properties and optionally one or more acceptable excipients;
c. an oral composition, either liquid or solid, comprising at least one carbohydrate compound, water at least one ingredient having antioxidant properties and optionally one or more acceptable excipients, wherein the antioxidant composition is substantially free of amino acids; and
instructions for the administration.

12. The oral liquid composition according to claim 5, wherein the ingredient having antioxidant properties is selected from the group consisting of flavonoids, ellagitannins, xanthones, tannins, anthocyanins, Vitamin C, Vitamin E, indole-3-carbinol, glutathione, and mixtures thereof.

13. A method for the treatment or prevention of postpartum blues or depression in a subject in need thereof, the method comprising administering the oral liquid composition as defined in claim 1, comprising a therapeutically effective amount of one amino acid selected from L-tyrosine and L-tryptophan, the at least one carbohydrate compound, water and one or more pharmaceutically acceptable excipients.

14. The method of claim 13, which comprises administering an oral liquid composition wherein the amino acid is L-tryptophan, and an oral liquid composition wherein the amino acid is L-tyrosine.

15. (canceled)

16. The method of claim 13, wherein the tryptophan oral liquid composition is administered on day-4 postpartum and the tyrosine oral liquid composition is administered on day-5 postpartum.

17. The method of claim 13, which further comprises administering an oral composition, either solid or liquid, comprising an antioxidant compound.

18. The method of claim 17, wherein the further oral composition comprising the antioxidant compound is administered days-3 and -4 postpartum.

19. (canceled)

20. The kit of claim 10, for treatment or prophylaxis of postpartum blues or depressed mood in a subject in need thereof, the kit comprising:
(a) the oral liquid composition comprising about 2 g of L-tryptophan per serving and from 50-60 g of simple carbohydrate, the at least one carbohydrate compound, water and one or more pharmaceutically acceptable excipients; and
(b) one or more servings of the oral liquid composition comprising about 10 g of L-tyrosine per serving and from 50-60 g of simple carbohydrate.

21. The kit of claim 20, comprising a plurality of liquid compositions together with instructions for oral administration in a multi-day postpartum regime for treatment or prophylaxis of postpartum blues or depressed mood in a subject in need thereof, said plurality of liquid compositions comprising:
a first composition and a second composition comprising 50-60 g carbohydrate and an antioxidant source, wherein the carbohydrate is from 5%-30% w/w with respect to the total liquid weight of the composition, wherein the first and second composition are essentially free of amino acids and may be the same or different from each other;
a third composition comprising 50-60 g carbohydrate, an antioxidant source, and an amino acid consisting of L-tryptophan, wherein: the weight ratio of L-tryptophan to carbohydrate is from 1:10 to 1:40, and the carbohydrate is from 5%-30% w/w with respect to the total liquid weight of the composition; and
a fourth composition comprising 50-60 g carbohydrate, an antioxidant source, and an amino acid consisting of L-tyrosine, wherein: the weight ratio of L-tyrosine to carbohydrate is from 1:2 to 1:30, and the carbohydrate is from 5%-30% w/w with respect to the total liquid weight of composition; and
instructions for administration of:
the first composition on the evening of the first day of the postpartum regime;
the second composition on the morning of the second day of the postpartum regime;
the third composition on the evening of the second day of the postpartum regime; and
the fourth composition on the morning of the third day of the postpartum regime.

22. The kit of claim 21, wherein:
the third composition comprises a weight ratio of L-tryptophan to carbohydrate of from 1:20 to 1:35 or from 1:25 to 1:30; and
the fourth composition comprises a weight ratio of L-tyrosine to carbohydrate of from 1:3 to 1:15 or from 1:4 to 1:10.

23. The kit of claim 21, wherein:
the first day of the regime corresponds with the subject's postpartum day 3;
the antioxidant source comprises blueberry extract and/or blueberry juice;
the carbohydrate is from 20%-30% w/w of the total weight of the composition;
the third composition comprises from 1-5 g of L-tryptophan, or 2 g L-tryptophan;
one or more of the compositions is in provided in the form of a shake;
the carbohydrate comprises simple carbohydrates, or sucrose;
the fourth composition comprises from 5-20 g of L-tyrosine, or 10 g L-tyrosine;
the antioxidant source comprises a blueberry extract and/or juice providing 6500 ORAC units/g; and/or
the compositions further comprise one or more excipient or vehicle selected from the group consisting of taste-masking agents, bitter blocker agents, sweeteners, flavorings, viscosity control agents, thickeners, guar gum, fiber, and cocoa powder.
